(19) United States
(12) Patent Application Publication
(10) Pub. No.: US 2023/0013550 A1
(43) Pub. Date: Jan. 19, 2023

(54) END EFFECTOR IDENTIFICATION IN SURGICAL ROBOTIC SYSTEMS

(71) Applicant: DePuy Synthes Products, Inc., Raynham, MA (US)

(72) Inventors: Robert Brik, Brookline, MA (US); Tarik Yardibi, Wayland, MA (US); William J. Frasier, New Bedford, MA (US); Marc Puls, Thörigen (CH); Brice Dudley, JR., Round Rock, TX (US)

(21) Appl. No.: 17/376,508

(22) Filed: Jul. 15, 2021

(51) Int. Cl. A61B 34/00; A61B 34/32; A61B 90/96

(52) U.S. Cl. CPC A61B 34/25; A61B 34/32; A61B 90/96; A61B 2034/256

(57) ABSTRACT

Systems, methods, and devices are disclosed for end effector identification in robotic surgical systems. A surgical robot can be coupled to an end effector. The system can identify the end effector using data received from the end effector. The system can adjust operation of the surgical system, including the robot arm, based on the data received from the end effector. Data received from or regarding the end effector can include detected characteristics, retrieved characteristics, or data stored on the end effector and communicated to the system. Both the identification and the operation adjustments can be performed automatically such that the system experiences little to no lag or downtime when coupling with different end effectors.

### BACKGROUND

Maintenance of a sterile surgical field is important to reduce the risk of patient infection or other complications. With the increasing prevalence of systems for performing robotic or robot-assisted surgeries in modern-day operating rooms, it is becoming increasingly challenging to effectively and efficiently maintain a sterile work environment, e.g., without compromising speed and efficiency of the procedure. For example, robot-assisted surgical procedures can involve multiple tools (e.g., used in association with instruments), used individually or in tandem, to perform the procedure, and can require a seamless transition between the same. Robotic-assisted surgical systems (e.g., referred to herein as robotic surgical systems for brevity) can have a robot arm for precisely positioning the tool. In practice, an end effector is interposed between a distal end of the robot arm and the tool, such as for holding the tool. Transitions from end effector to end effector, from tool to tool, or replacing instruments held by a tool, must occur without introducing possible contaminants into the surgical environment. For more complex surgeries, many transitions are planned in the course of the procedure. In some cases, a large number of components need to be made available during the surgery, causing the surgeon to frequently, and repeatedly, switch between several different tools, resulting in frequent insertion, removal, and re-insertion of instruments to and from the surgical site.

Existing methods for switching tools during surgery have several shortcomings. For example, one benefit of robotic surgical systems is their positioning and navigation functionalities, however, in the course of disconnecting one tool and connecting another, the tool, or in some cases the entire system, may need to be recalibrated to adjust to the dimensions of the new tool. Tools that are of different weights, sizes, and shapes can alter loads on the system, such as the robot arm, prompting the system to recalibrate to ensure that precision is maintained. Such recalibrations can slow down surgeries by having to reset the relative coordinates of the system, the tool, and the patient, among others, increasing the time spent in the operating room, and minimizing the number of patients that can be treated daily. Moreover, frequent recalibration can introduce user error and failure to accurately recalibrate coordinates can result in undesirable outcomes. Further, recalibration procedures are typically performed manually, which introduces surgery lag times and increases user interface with the tools, which can compromise sterility of the surgical space. Lastly, manual tracking of performance and lifecycle of various tools can compromise operating room resources, while failure to keep accurate records can lead to suboptimal surgical outcomes.

Still further, in some cases a robotic surgical system controller can be pre-programmed with operating parameter information for every end effector or tool envisioned as part of the system. This can introduce complications, however, as a manufacturer might make a last-minute design change that alters various characteristics of an end effector, or there might be evolution of an end effector's design over a series of batches created. In a conventional system, any such change would require a reprogramming of the robotic surgical system controller, which can be cumbersome, time-consuming, and expensive.

Accordingly, there is a need for systems, devices, and methods for end effector identification in robotic surgical systems that provide a more flexible system where a controller can identify an end effector based on data received from the end effector. Further, there is a need for systems, devices, and methods where data received from the end effector can include parameters that can be used to adjust operation of the robotic surgical system to optimize for the detected end effector.

### SUMMARY

Systems, methods, and devices are disclosed for end effector identification. For example, a robotic surgical system is disclosed comprising a robot arm, a controller operably connected to the robot arm for positioning a tool with respect to a patient, wherein the tool is retained by an end effector, the controller being configured to: determine whether the end effector is coupled to the robot arm; receive data from the end effector; identify the end effector; and adjust operation of the surgical system (for example, the robot arm) based on the data received from the end effector. In another example, an end effector is disclosed comprising a memory for storing data about the end effector, and a processor configured to send the data to a controller for a robotic surgical system. In another example, a surgical method is disclosed comprising coupling an end effector to a robot arm that is coupled to a controller, identifying the end effector with the controller using data received from the end effector, and adjusting operation of the surgical system (for example, the robot arm) with the controller based on the data received from the end effector.

### BRIEF DESCRIPTION OF THE DRAWINGS

FIG. 1 is a flowchart of using an end effector according to the present disclosure;

FIG. 2 is a flowchart of end effector identification and uses of data received from the end effector;

FIG. 3 is a schematic of a system including a surgical robot and an end effector;

FIG. 4 depicts an end effector and a tool retained by the end effector;

FIG. 5 depicts another end effector and a tool retained by the end effector;

FIG. 6 depicts another end effector and a tool retained by the end effector;

FIG. 7 depicts an end effector that includes device for storing parameters;

FIG. 8 is a diagram of a device for storing end effector parameters; and

FIG. 9 depicts an end effector having an integrated parameter storage device and coupled to a robot arm.

### DETAILED DESCRIPTION

Systems, devices, and methods for end effector identification in robotic surgical systems are provided herein. In particular, the systems, devices, and methods disclosed herein allow for automatic identification of a particular end effector by a robotic surgical system based on data received from and/or regarding the end effector. Data received from or regarding the end effector can include detected characteristics, retrieved characteristics, or data stored on the end effector and communicated to the system. Such data can include general characteristics of the end effector, such as dimensions, shape, weight, center of mass, or other mechanical parameters or characteristics, as well as any of a variety of more specific data parameters, such as the location, function, history (e.g., lifetime, service, and/or sterilization), and other parameters of the end effector. Data can be stored on the end effector and communicated to the robotic surgical system automatically (e.g., upon a trigger, such as in connection with coupling the end effector to the robot arm). Alternatively, the end effector can be prompted by the robotic surgical system to send the data, such as after coupling. Data received from the end effector can cause the system to query databases for additional information about the end effector.

Identification of the end effector can be performed automatically such that the system experiences little to no lag or downtime. The system can identify the end effector via embedded circuitry on the end effector. Additionally, additional data can be determined using optical recognition via visualization cameras and/or a navigation system (e.g., to detect shape or dimensions), or by using internal sensors of the robot arm (or system) to detect characteristics of the end effector (such as weight, center of mass, power draw, torque required to actuate, etc.). The end effector can store parameter data thereon that is configured to be identified by the system. The system can be configured to adjust its operation based on the parameters. For example, the system can track parameters such as location, dimensions, lifetime, service, and/or sterilization history of the end effector, among others. In some embodiments, the system can recognize the end effector and attach thereto, while system software can automatically utilize a module that corresponds to the respective end effector to control said end effector.

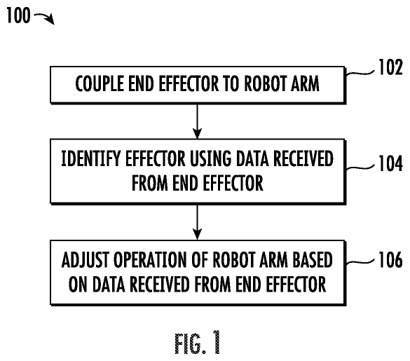

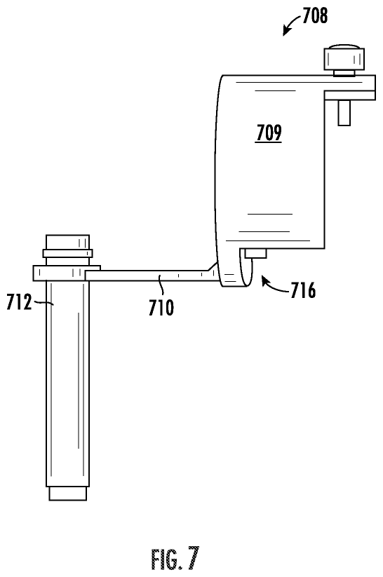

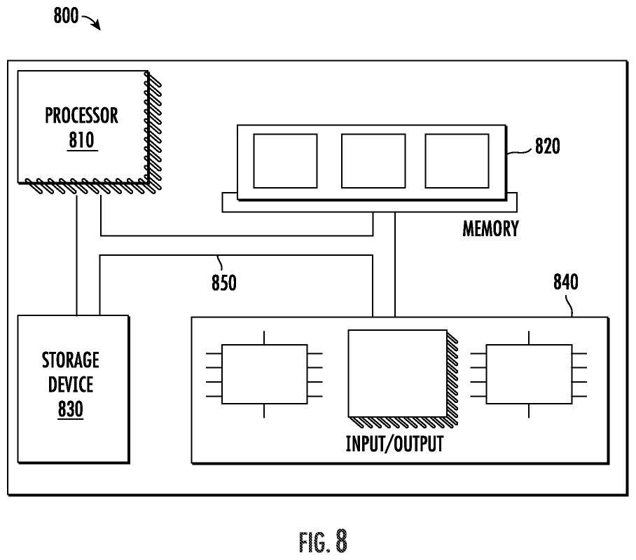

FIG. 1 illustrates a method 100 of end effector identification. Discussion of this method is illustrative and not intended to limit the disclosure. For example, portions of the flowchart are referred to as steps, but it is understood that the steps can be omitted, repeated, sub-divided, concurrently performed, or performed in a different order without departing from the spirit of the disclosure. At step 102, an end effector is coupled to a robot arm. Both the end effector and the robot arm can be part of a robotic surgical system. It is understood that the coupling is at least partially mechanical in nature, but the system can receive input that the end effector is being attached, or alternatively, the system can instruct a user to attach the end effector. The coupling can be detected by the system, for example, by completion of an electrical circuit (such as will be described with respect to the contacts 716 in FIG. 7).

At step 104, the end effector communicates data to the system, and the system identifies the end effector (e.g., using data received from or regarding the end effector). Examples of data include a unique identifier, a version identifier, a class identifier, manufacturing information, shape, a dimension, weight, center of mass, duration of use, lifetime information, service history, or sterilization history. Detection and identification of an end effector can be completed automatically, e.g., upon presentation of an end effector in a camera field of view, coupling the end effector to the robot arm, etc., or can be performed at the request of a user, e.g., when a user initiates a detection and identification process via an input such as a button, etc. In some embodiments, the end effector sends a unique identifier, and the system uses the unique identifier to query a database and obtain data regarding the end effector.

At step 106, the system adjusts operation of the robot arm (e.g., based on data received from the end effector). Examples of adjustments include an adjustment to the robot arm based on an estimated load and/or force exerted by the end effector weight or center of mass, an adjustment to the robot arm to allow for optimal joint configuration of the robot arm based on tool trajectory, or an adjustment to calibration data.

Any of a variety of alternative or additional steps are possible in connection with the method shown in FIG. 1. For example, steps 102 and 104 can be reversed, such that the robotic surgical system can be configured to identify the end effector prior to coupling the end effector to the robot arm. A robotic surgical system can be configured to further identify an end effector by detecting its shape using one or more cameras connected to the system. In such an embodiment, one surgical workflow might include a user presenting the end effector in front of a system tracking unit or a system camera for identification. Some data regarding the end effector can be detected characteristics of the end effector, such as its weight, center of mass, torque required to actuate, electrical draw, or other forces or loads placed on the robot arm upon coupling. Data received from the end effector can be stored on a memory that is coupled to or integrated into the end effector. The stored data can be actively communicated to a robotic surgical system (e.g., a robot controller, etc.) from the end effector. Such communication can include wired or wireless communication over a variety of protocols, such as wireless (e.g., near-field communication (NFC), WIFI™, BLUETOOTH™, BLUETOOTH LE™, ZIGBEE™, and the like) or wired (e.g., USB or Ethernet).

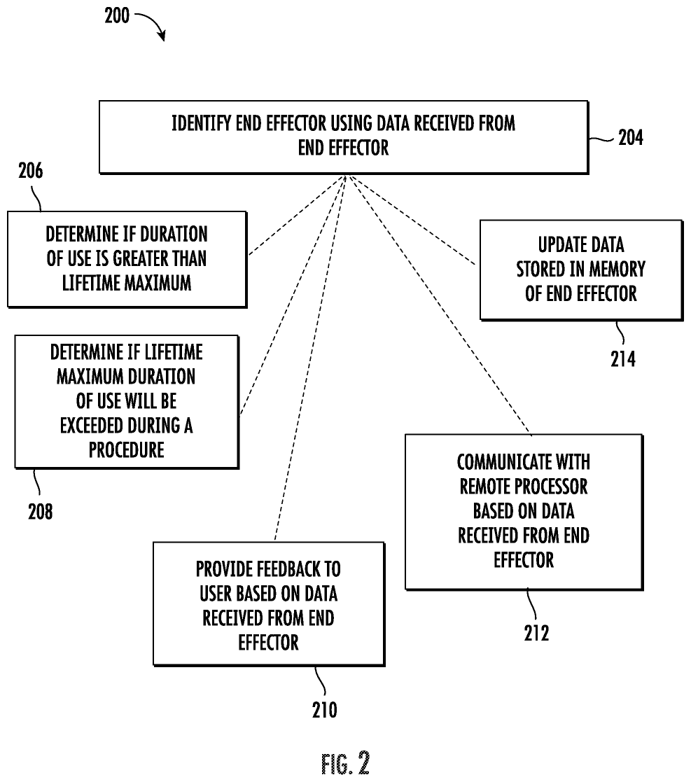

FIG. 2 illustrates a method 200 of end effector identification and use of data received from the end effector, for example, in conjunction with a robotic surgical system or a surgical plan. As shown with respect to FIG. 1, an end effector can (e.g., first) be coupled to a robot arm. Both the end effector and the robot arm can be part of a robotic surgical system. It is understood that at the coupling includes mechanical attachment, and can also include the system receiving input that the end effector is being attached (such as by sensing with peripheral devices, completion of an electrical circuit between the end effector and robot arm, or receiving instructions from the controller). Alternatively, the system can instruct a user to attach the end effector, and can require confirmation from the user after attachment. In yet another alternative, the end effector is identified before coupling.

At step 204, the end effector communicates data to the system, and the system identifies the end effector (e.g., using data received from or regarding the end effector). A plurality of steps 206-214 can be implemented in connection with using data received from or regarding the end effector. Many of these steps would not result in an adjustment to a robot arm, but would be useful to a surgical plan that can be carried out using, for example, a robotic surgical system. It is understood that not all of steps 206-214 need to be performed, and all combinations and sub-combinations (e.g., one or more, two or more, three or more, four or more, five or more) are contemplated, and then, in any order, and as such, they are illustrated in a fan shape, with dotted line connectors.

At step 206, a determination (e.g., by the controller based on the data) is made if a duration of use of the end effector is greater than a lifetime maximum. In such an embodiment, any of a lifetime total use and prescribed lifetime maximum use can be included in data received from the end effector.

At step 208, a determination (e.g., by the controller based on the data) is made if a lifetime maximum duration of use will be exceeded during a procedure, such as a current or upcoming procedure. In such embodiments, any of a lifetime total use and prescribed lifetime maximum use can be included in data received from the end effector, and can be combined with data accessible to the robotic surgical system such as planned time of use for a current or upcoming procedure.

At step 210, a provision of feedback is made (e.g., by the controller based on the data) to a user based on data received from the end effector. Such feedback can include any of auditory, haptic, and visual feedback (e.g., visual feedback using a system display). Feedback can include conveying information, such as a unique identifier of the end effector or other information associated with the end effector, such as history of couplings with robot arms, service history, sterilization history or status, etc. Feedback can also include warnings to a user, e.g., if a lifetime maximum time of use has been reached or is close to being reached (e.g., as determined at step 208), if sterilization has not been performed, if there are diagnostic errors, etc.

At step 212, a communication, such as with a remote processor, is made based on data received from the end effector. For example, the system can receive data from the end effector that includes a unique identifier and can then access one or more remote processors, databases, etc., to pull additional information associated with the unique identifier and end effector. The additional information can be any of the information mentioned above, e.g., service history, sterilization history, lifetime maximum use, manufacturer updates, etc. In some embodiments, steps 210 and 212 are both performed, and in any order such as step 212 and then step 210.

At step 214, an update is performed, such as updating data stored in memory of the end effector. In embodiments where the end effector includes a memory to store data that can be communicated (e.g., to the system upon coupling), the memory can in some embodiments be writable such that the information stored on the memory can be updated following use during a procedure. For example, in some embodiments a lifetime total use value can be updated after use with the system to reflect the additional time the end effector was used since the end effector was first coupled to the robot arm. A number of procedures or times the end effector is used may be incremented in memory (e.g., after each attachment or use). In addition, other parameters such as sterilization history, service updates, etc. can be updated in a similar manner. Such updates can be created by the system tracking various parameters and writing updated data to a memory on the end effector after use (e.g., prior to, or otherwise in connection with, decoupling the end effector from the robot arm) or by a processor integrated into the end effector tracking such data and writing updates to the memory (e.g., independent of any system instruction). In some embodiments, updates can alternatively or additionally be written to one or more remote data stores, e.g., such as one or more databases or other data stores connected to the system via one or more network connections (e.g., cloud-based storage, etc.). In this manner, parameters such as sterilization history, use, service updates, etc., can be monitored remotely and available to a variety of users at different locations. After the system identifies the end effector (e.g., using data received from the end effector), the system can adjust operation of the system (e.g., based on data received from the end effector or on additional data retrieved by the system controller (e.g., a database) after receiving the initial data from the end effector).

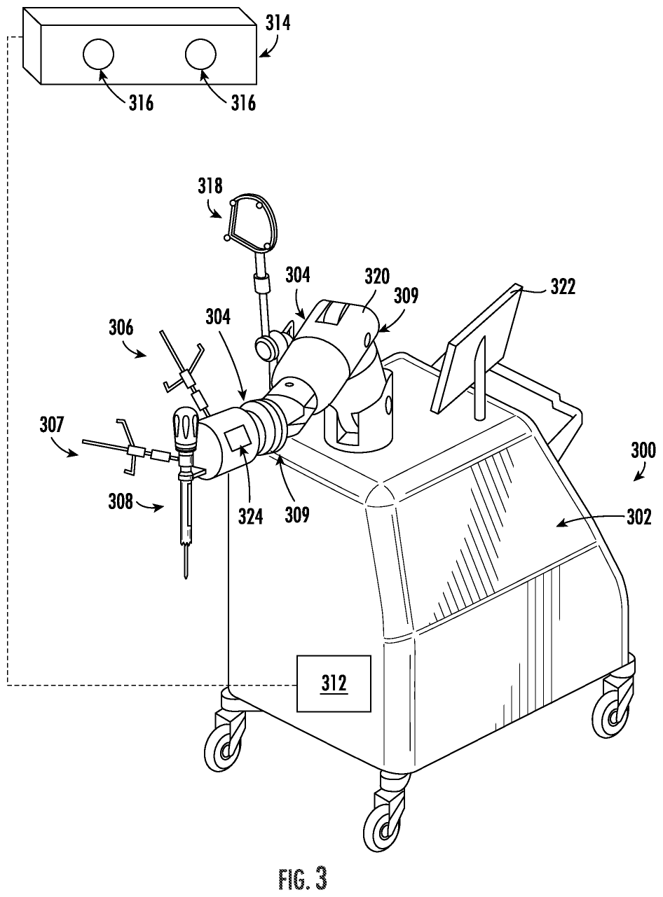

FIG. 3 shows an overview of a robotic surgical system 300 according to the present disclosure. A surgical robot base 302 supports a robot arm 320. The robot arm 320 includes a plurality of arm segments 304 connected by rotatable or otherwise articulating joints 309. The robot arm 320 can be moved by actuation of the joints, locked in place, etc. Rotation and/or flexion about the joints 309 can allow the robot arm 320 to move in all six degrees of freedom during a procedure. The joints 309 can be configured for incremental changes in each of the six degrees of freedom to ensure precision in operation of a plurality of different end effectors during surgery. The robot arm 320 can move about the joints 309 to position arm in a desired position relative to the patient. A control unit or controller 312 controls the actuation of each joint 309 in order to position the robot arm 320. The controller 312 typically includes a power supply, AC/DC converters, motion controllers to power the motors of the actuation units in each joint, fuses, real-time interface circuits, and other components conventionally included in robotic surgical systems.

An external device 322 can communicate with the controller 312. The device 322 can be a display, a computing device, remote server, etc., configured to allow a surgeon or other user to input data directly into the controller 312. Such data can include patient information and/or surgical procedure information. The device 322 can display information from the controller, such as alerts. Communication between the device 322 and the controller 312 can be wireless or wired.

An end effector 308 can be coupled to the robot arm 320. The system 300 can utilize end effectors of various shapes, sizes, and functionalities. In some embodiments, the robot arm 320 can have the end effector 308 on a distal end thereof, as shown, although the end effector can couple to the robot arm along a length thereof in other embodiments. The end effector 308 can retain a tool (e.g., used in association with instruments) (not numbered). The system 300 (e.g., via controller 312) can control a position of the end effector 308 via a position of the robot arm 320 to perform surgical procedures. The system 300 (e.g., via controller 312) can have a wired attachment to the end effector 308 and/or the system can communicate with the end effector wirelessly. The robot arm 320 that connects to the end effector can have a variety of configurations that support robot-assisted surgery.

The robot arm 320 can include one or more features for mating with counterpart mating features of the end effector 308. These additional mating features can aid with alignment of the components and can increase stability of a coupling between the robot arm 320 and the end effector 308. For example, in some embodiments, a distal face of the robot arm 320 can include one or more pin recesses (not shown), which can act as female mating features that can align with and receive counterpart male mating features of the end effector 308 (e.g., contacts 716 shown in FIG. 7). Alternative arrangements of the mating features are within the scope of the present disclosure. For example, the robot arm 320 can include male mating features with counterpart female mating features on the end effector 308. Moreover, placement and number of such features can be varied. In the case of the robot arm 320, the mating features can be positioned on a distal end thereof for engaging the end effector 308. The mating features can complete a circuit when engaged, thus indicating coupling between the robot arm 320 and the end effector 308.

The system 300 can utilize one or more sensors (not shown) thereon for identifying the end effector 308 attached thereto. For example, in some embodiments, the controller 312 can utilize internal torque, force, or other sensors to identify the end effector 308 by its center of mass, weight, or another mechanical parameter associated with the end effector. The controller 312 can receive various other parameters from the end effector, such as time in use, sterilization history, etc. In turn, each end effector 308 can be configured for detecting the system 300 and communicating data parameters of the end effector to the system.

A navigation array 306 can be mounted on the robot arm 320 to determine a position of the end effector 308. The structure and operation of the navigation array 306 can vary depending on the type of navigation system used. In some embodiments, the navigation array 306 can include one or more sphere-shaped or other fiducials for use with an optical navigation system, for example, a robotic navigation system. The navigation system can facilitate registering and tracking the position and/or orientation of the navigation array 306 and, by extension, the end effector 308 and its relative distance to other objects in the operating room, e.g., a patient, a surgeon, etc. In some embodiments, a navigation array 307 can be directly attached to the end effector 308. In some embodiments, the array 307 can be embedded in a surface of the end effector 308. In some embodiments, a navigation array 318 can be coupled to another component of the robotic device, such as the base 302 of the robotic arm 320. Fiducials of the array 306, 307, or 318 can be arranged in predetermined positions and orientations with respect to one another. The fiducials can be aligned to lie in planes of known orientation (e.g., perpendicular planes, etc.) to enable setting of a Cartesian reference frame. The fiducials can be positioned within a field of view of a navigation system and can be identified in images captured by the navigation system. Exemplary fiducials include infrared reflectors, light emitting diodes (LEDs), spherical reflective markers, blinking LEDs, augmented reality markers, and so forth. The navigation arrays can be or can include an inertial measurement unit (IMU), an accelerometer, a gyroscope, a magnetometer, other sensors, or combinations thereof. The sensors can transmit position and/or orientation information to a navigation system, e.g., to a processing unit of the navigation system, which can, for example be the controller 312.

The location of the end effector 308 can be calculated by receiving a position signal from an encoder in each joint 309 and/or by measuring a position of one or more of the above-described navigation arrays to directly detect the position. The end effector 308 retains a tool (not numbered) that retains an instrument or surgical device, for example, a drill, scalpel, saw blade, burr, reamer, mill, knife, or any other implement that could cut or deform bone and is appropriate for use in a given operation. Further, the present disclosure is also contemplated to include use of such instruments by surgical robots, by users with some degree of robotic assistance, and without involvement of surgical robots or robotic assistance (e.g., once positioned).

The system 300 can also comprise a tracking unit 314, such that the relative pose or three-dimensional position and orientation of the navigation array 306 (and/or other navigation arrays) can be tracked in real time and shared to the controller 312 for planning or control. The tracking unit 314 can measure the relative motions between any and all components coupled to navigation arrays in a known manner. Tracking can be performed in a number of manners, e.g., using stereoscopic optical detectors 316, ultrasonic detectors, sensors configured to receive position information from inertial measurement units, etc. Tracking in real time can, in some embodiments, mean high frequencies greater than twenty Hertz, in some embodiments in the range of one hundred to five hundred Hertz, with low latency, in some embodiments less than five milliseconds. Regardless of how it is gathered, position and orientation data can be transferred between components (e.g., to the controller 312) via any suitable connection, e.g., with wires or wirelessly using a low latency transfer protocol. The real-time controller 312 can carry out real-time control algorithms at a reasonably high frequency with low additional latency to coordinate movement of the system 300. The tracking unit can also include cameras, or use the stereoscopic optical detectors 316, to detect, for example, a shape and/or a dimension of the end effector 308.

The end effector 308 can include a unique identifier 324. The unique identifier can be passive, such as a QR code, bar code, serial number, ID number, or other marking or tag that uniquely identifies a particular end effector (e.g., or a particular group of end effectors). The unique identifier 324 can be active, such as an RFID tag readable by a sensor of the system 300 or another data transmission technique. The system 300 can recognize the unique identifier 324 (by way of a non-limiting example, in captured images of the end effector 308) and determine further information from said unique identifier information. For example, the unique identifier 324 can serve as a lookup key which can be used by the system 300 to retrieve information from a database, such as a network connected server, cloud storage, and the like. Example information that can be retrieved by the system 300 based on the unique identifier 324 can include manufacturing information, end effector usage (e.g., if compliant with a single-use policy), history, service/replacement data, among others. In some embodiments, the unique identifier 324 can be embedded into the end effector 308.

FIG. 4 shows a guide end effector 408 that can be used to facilitate drilling, tapping, and screwing operations utilizing a variety of drills and drivers inserted through a guide cannula 412. The end effector 408 has a housing 409. A plurality of alignment markers 409a are disposed on the housing 409. Examples of alignment markers 409a include infrared reflectors, LEDs, spherical reflective markers, blinking LEDs, augmented reality markers, and so forth. Manufacturing information about the end effector 408 includes data about the pattern and tolerances associated with the alignment markers 409a. In some embodiments, this information can be used to adjust calibration data (e.g., using the marker position with pattern and/or tolerance information). In some embodiments, knowing exact end effector or tool geometry based on the manufacturing information can maintain and/or improve accuracy of the system.

An instrument mount 410 extends from the housing 409. The instrument mount 410 can hold the guide cannula 412 that receives any of a variety of instruments 414, e.g., a drill, tap, saw, or combination instrument, with trajectory A1 that is perpendicular relative to a longitudinal axis L1 of a distal portion of a robot arm (not depicted) that is attached to the end effector 408. The end effector 408 can have a unique identifier, be recognized by a robotic surgical system, and/or can send data. The system can, e.g., automatically, identify the particular end effector 408 based on data received from or regarding the end effector. Such data can include general characteristics of the end effector 408, such as dimensions, shape, weight, center of mass, or other mechanical parameters or characteristics, as well as any of a variety of more specific data parameters, such as the location, function, manufacturing information, history (e.g., lifetime, service, and/or sterilization), and other parameters of the end effector. Data can be stored on the end effector 408 and communicated to the robotic surgical system automatically (e.g., such as in connection with coupling the end effector to a robot arm). Alternatively, the end effector 408 can be prompted by the robotic surgical system to send the data, such as after coupling. Data received from the end effector 408 can cause the system to query databases for additional information about the end effector.

FIG. 5 shows an end effector 508 that can be used to facilitate drilling, tapping, and screwing operations utilizing a variety of drills and drivers inserted through a guide cannula 512. The end effector 508 has a housing 509. A plurality of alignment markers (not depicted) can be disposed on the housing 509. Manufacturing information about the end effector 508 can include data about the pattern and tolerances associated with the alignment markers. In some embodiments, this information can be used to adjust calibration data (e.g., using the position and tolerance information). An instrument mount 510 extends from the housing 509. The n instrument mount 510 can hold the guide cannula 512. The guide cannula 512 has a trajectory A2 that is non-perpendicularly angled or oblique relative to a longitudinal axis L2 of a distal portion of a robot arm (not depicted) that is attached to the end effector 508. The end effector 508 can have a unique identifier, be recognized by a robotic surgical system, and/or can send data. The system can, e.g., automatically, identify the particular end effector 508 based on data received from or regarding the end effector. Such data can include general characteristics of the end effector 508, such as dimensions, shape, weight, center of mass, or other mechanical parameters or characteristics, as well as any of a variety of more specific data parameters, such as the location, function, manufacturing information, history (e.g., lifetime, service, and/or sterilization), and other parameters of the end effector. Data can be stored on the end effector 508 and communicated to the robotic surgical system automatically (e.g., such as in connection with coupling the end effector to a robot arm). Alternatively, the end effector 508 can be prompted by the robotic surgical system to send the data, such as after coupling. Data received from the end effector 508 can cause the system to query databases for additional information about the end effector.

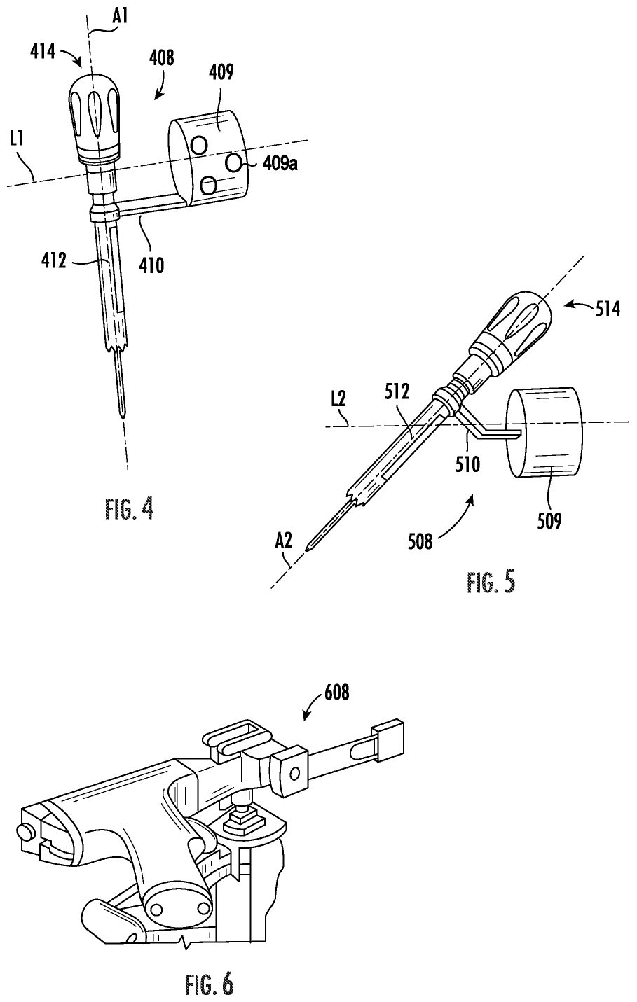

FIG. 6 illustrates an oscillating saw end effector 608 that can be used, for example, in a surgical procedure on a shoulder or knee. The end effector 608 can be coupled to a distal portion of a robot arm (not depicted). The end effector 608 can have a unique identifier, be recognized by a robotic surgical system, and/or can send data. The system can, e.g., automatically, identify the particular end effector 608 based on data received from or regarding the end effector. Such data can include general characteristics of the end effector 608, such as dimensions, shape, weight, center of mass, or other mechanical parameters or characteristics, as well as any of a variety of more specific data parameters, such as the location, function, history (e.g., lifetime, service, and/or sterilization), manufacturing information, and other parameters of the end effector. A plurality of alignment markers (not depicted) can be disposed on the end effector 608, and manufacturing information can include data about the pattern and tolerances associated with the alignment markers. In some embodiments, this information can be used to adjust calibration data (e.g., using the position and tolerance information). Data can be stored on the end effector 608 and communicated to the robotic surgical system automatically (e.g., such as in connection with coupling the end effector to a robot arm). Alternatively, the end effector 608 can be prompted by the robotic surgical system to send the data, such as after coupling. Data received from the end effector 608 can cause the system to query databases for additional information about the end effector.

FIG. 7 illustrates an end effector 708. The end effector 708 has a housing 709. An instrument mount 710 extends from the housing 709. A plurality of alignment markers (not depicted) can be disposed on the housing 709. Manufacturing information about the end effector 708 can include data about the pattern and tolerances associated with the alignment markers. In some embodiments, this information can be used to adjust calibration data (e.g., using the position and tolerance information). The instrument mount 710 can hold a guide cannula 712. The guide cannula 712 can receive a tool (not depicted) and/or an instrument (not depicted) and direct it toward a surgical site. The housing 709 retains a memory, processor, and/or other integrated electronics for communicating with a controller (not depicted), for example, after coupling to the robot arm. In some embodiments, the memory, processor, and/or other integrated electronics can include a wireless communication module to implement wireless communications between the end effector 708 and controller, e.g., for sending and receiving data. Data can be any described herein, and can be stored in memory. Contacts 716, extend from the housing 709. In some embodiments, the contacts 716 complete an electrical circuit when the end effector 708 is coupled to the robot arm, which can be used to detect coupling (e.g., by a controller of a robotic surgical system). In some embodiments, the contacts 716 establish wired communication between the end effector 708 and the robot arm, e.g., for sending and receiving data.

FIG. 8 is a block diagram of a device 800 that can be integrated into end effectors of the present disclosure. The device 800 can include any combination of a processor 810, a memory 820, a storage device 830, and an input/output device 840. Each of the components 810, 820, 830, and 840 can be interconnected, for example, using a system bus 850. The processor 810 can be capable of processing instructions for execution within the device 800. The processor 810 can be a single-threaded processor, a multi-threaded processor, or similar device. The processor 810 can be capable of processing instructions stored in the memory 820 or on the storage device 830.

The memory 820 can store information within the device 800. In some embodiments, the memory 820 can be a computer-readable medium. The memory 820 can, for example, be a volatile memory unit or a non-volatile memory unit. In some embodiments, the memory 820 can store information related to various surgical robots, robot arms, and navigation arrays, among other information.

The storage device 830 can be capable of providing mass storage for the system 800. In some embodiments, the storage device 830 can be a non-transitory computer-readable medium. The storage device 830 can include, for example, a solid-date drive, a flash drive, and/or some other larger capacity storage device. The storage device 830 can alternatively be a cloud storage device, e.g., a logical storage device including multiple physical storage devices distributed on a network and accessed using a network. In some embodiments, the information stored on the memory 820 can also or instead be stored on the storage device 830.

The input/output device 840 can provide input/output operations for the device 800. In some embodiments, the input/output device 840 can include one or more of network interface devices (e.g., an Ethernet card), a serial communication device (e.g., an RS-232 10 port), and/or a wireless interface device (e.g., a short-range wireless communication device, an 802.11 card, a 3G wireless modem, a 4G wireless modem, a 5G wireless modem). In some embodiments, the input/output device 840 can include driver devices configured to receive input data and send output data to other input/output devices, e.g., a keyboard, a printer, and/or display devices (such as associated with a robotic surgical system).

In some embodiments, the device 800 can be a microcontroller. A microcontroller is a device that contains multiple elements of a computer system in a single electronics package. For example, the single electronics package could contain the processor 810, the memory 820, the storage device 830, and/or input/output devices 840.

As noted above, in some embodiments the end effectors can have one or more parameters stored thereon. For example, each end effector can include one or more features associated therewith that communicate information about the surgical procedure to the system 300 (FIG. 3). For example, an end effector (such as shown in FIGS. 4-7) can include the device 800 to store parameter data that can be accessed prior to, during, or after the surgical procedure. In some embodiments, the system can log data about the parameters for tracking and use of the system that can be used to optimize the system.

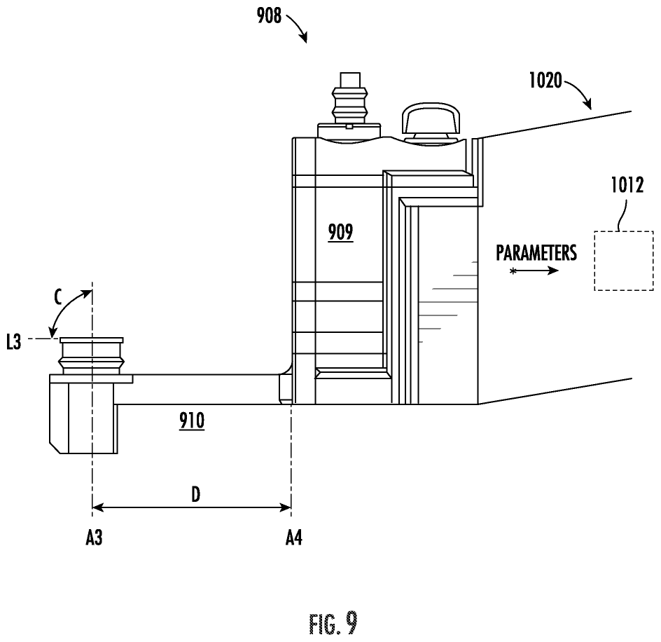

FIG. 9 illustrates an end effector 908. The end effector 908 has a housing 909. An instrument mount 910 extends from the housing 909. A plurality of alignment markers (not depicted, but such as illustrated and described with respect to FIG. 4) can be disposed on the housing 909. Manufacturing information about the end effector 908 can include data about the pattern and tolerances associated with the alignment markers. In some embodiments, this information can be used to adjust calibration data (e.g., using the position and tolerance information).

The instrument mount 910 can hold a guide cannula 912. The guide cannula 912 can receive an instrument (not depicted) and direct it toward a surgical site. The housing 909 retains a memory, processor, and/or other integrated electronics (such as the device 800 of FIG. 8) for communicating with a system controller 1012 (e.g., of a robotic surgical system) of a robot arm 1020, such as after coupling thereto. In some embodiments, the electronics can include a wireless communication module to implement wireless communications between the end effector 908 and the controller 1012 for controlling the robot arm 1020, e.g., for sending and receiving data. Contacts (not visible) can extend from the housing 909 and complete an electrical circuit when the end effector 908 is coupled to the robot arm 1020. The contacts can be used to detect coupling (e.g., by the controller 1012). In some embodiments, the contacts establish wired communication between the end effector 908 and the robot arm 1020, e.g., for sending and receiving data.

After coupling of the end effector 908 to the robot arm 1020, parameters, e.g., data about the end effector that can be stored on the end effector, is communicated to the controller 1012. Data received from the end effector 908 can cause the controller 1012 to query databases for additional information about the end effector. For example, the system can have specific model information and specifications stored for various types (e.g., models) of end effectors. The end effector 908 can send data identifying its type or model. The controller 1012 can retrieve information indicating certain dimensions associated with the type or model. For example, such that the robot arm 1020 can detect and attach to the end effector 908 in a proper configuration). Alternatively, the dimensions can be used to calibrate a position of a center line of a tool (not depicted). A distance (D) between a trajectory A3 of the tool from an axial axis A4 of the distal end of the robot arm 1020 can be retrieved by the controller 1012. An angle (C) of the end effector trajectory A3 relative to a longitudinal axis L3 of the distal end of the robot arm 1020 can be retrieved by the controller 1012.

In some embodiments, parameters regarding the end effector 908 can be detected (e.g., by the controller 1012) using optical recognition via one or more cameras associated with the system. For example, the optical recognition cameras discussed above can be used not only to determine a location and/or a position of the end effector prior to coupling thereto, but read the parameters of the end effector prior to coupling. The end effector 908 has a size, weight, center of mass, and position. For example, once the coordinates of the end effector 908 are detected, the system can move the robot arm 1020 towards the end effector for coupling.

In some embodiments, the end effector 908 can store dimensional parameters thereon. For example, in some embodiments, the end effectors can store such dimensional information as weight and center of mass parameters onboard as data, e.g., in memory. Storage of such parameters as data on the end effector enables the system to read the end effector 908 when connected thereto for automatic updates of the system. Moreover, when end effector parameters are changed, the parameters can be read by the system to allow the system to update and adjust as needed. One example of such an adjustment can include compensating for various masses and center of masses of the end effector when the robot arm performs tasks, e.g., approaches a surgical site. Weight and/or center of mass information can be entered at a last stage of end effector manufacture to communicate these values to the rest of the system. In addition to location and dimensional parameter data, some non-limiting examples of parameters that can be stored on the end effectors can include sterilization methods and cycles, lifetime, service, and sterilization history of end effector, among other things.

Storing weight and/or center of mass information onboard the end effector can allow the system to sense and adjust to each end effector coupled thereto. Moreover, when the system connects to the end effector 908, the system can be configured to read the information on the end effector and update and/or adjust calibration data to include the parameters of the end effector. For example, the system can include one or more modules (not shown) thereon that correspond to each end effector, with the modules being configured to read end effector parameters. Once an end effector 908 is connected to the system, the system can detect the end effector and activate the corresponding operative module. For example, when the system detects that an instrument is retained by the guide 912, a module that corresponds to the instrument can be activated to read the parameters associated with the instrument, such as distance (D) or angle (C). In some embodiments, this information can be used to adjust calibration data (e.g., using the position and tolerance information). In some embodiments, this information can be used in accompaniment with alignment markers to adjust calibration data (e.g., using the position and tolerance information). These parameters can then be passed on to the system to perform adjustments. Automatic adjustments of the system can greatly decrease operating room time and eliminate the need to manually update system parameters and calibrations, which results in more efficient surgeries.

Several different adjustments can be made by the system in view of accessed parameters. For example, the system can compensate for various masses and centers of mass of a given end effector 908 to ensure that the system is balanced and maintains precision. In some embodiments, the system can include optimized robotic controls in which the system can adjust control algorithms based on estimated loads and/or forces exerted by a specific end effector. Performing these adjustments can result in smoother and more reliable operation of the system.

In some embodiments, the end effector 908 can store manufacturing information thereon, e.g., in memory. Alternatively, the manufacturing information can be on the label or placed on the end effector itself. The manufacturing information can include the manufacturer, batch number, part number, and function of the end effector, among other things, or can prompt the controller 1012 to search for manufacturing information. The system can use this information to provide a warning or indicate for the need for a modification based on the batch information. That is, in some embodiments, the system can provide a warning or alert based on the type of end effector 908 coupled thereto. For example, if the end effector 908 includes a cutting instrument, the system can provide a warning that the instrument needs sharpening or that its trajectory has veered off its target at the surgical site. It will be appreciated that the warning can be produced from one or more of the controller 1012, the end effector 908, or another piece of equipment, e.g., the navigation system. In some embodiments, the system can display errors or warning messages associated with the system by projecting such information onto a graphical user interface, via audio feedback, via haptic feedback, or the like. In other embodiments, information such as errors or warnings can be conveyed to a user by projecting patterns or other indicators around the surgical site to ensure notification.

In some embodiments, the end effector 908 can store dimensional constrains thereon. As with other parameters, storing dimensional constraints can allow the system to update when end effector parameters are changed, thereby preventing the need for updating system parameters manually. For example, the end effector 908 can store working volume parameters thereon such that the system can determine the length, width, and depth of the overall system when the end effector is attached thereto. Knowledge of the working volume can allow the system to be aware of the extents of the end effector so the system and its components can be prevented from colliding with other known volumes or other objects within the operating room, e.g., the patient, surgical apparatuses, other robots operating on the patient, walls of the operating room, and so forth. Knowledge of the exact unique dimensional data of each end effector can also increase system accuracy.

Additional non-limiting examples of parameters that the end effector 908 can store include storing the number of procedures the end effector is used in, names of systems to which the end effector has been attached, length of time the end effector 908 was in use, and the like. Once attached, the end effector 908 can also communicate lifetime constraints to the system and allow the system to determine whether the lifetime will be exceeded during the current procedure such that replacement will be needed. In some embodiments, the end effector 908 can also provide information about whether the lifetime will expire within the next n number of procedures.

In some embodiments, the end effector 908 can store information relating to a surgical procedure thereon. Such information can include, for example, information related to working direction and/or trajectory of an end effector and/or robot arm that allows for optimal joint configurations and/or break configurations. For example, if the end effector includes a tube as an instrument guide 912, etc., this can result in a certain preferred direction and/or trajectory for robot movement to accommodate the positioning of the end effector 908. Similar to the example above, such information can be utilized to provide optimized operation of the system.

Examples of the above-described embodiments can include the following.

In a first example, a robotic surgical system comprises a robot arm; a controller operably connected to the robot arm for positioning a tool with respect to a patient, wherein the tool is retained by an end effector, the controller being configured to: determine whether the end effector is coupled to the robot arm; receive data from the end effector; identify the end effector; and adjust operation of the surgical system (for example, the robot arm) based on the data received from the end effector. In some embodiments, the data received from the end effector is at least one of a unique identifier, a version identifier, a class identifier, manufacturing information, shape, a dimension, weight, center of mass, duration of use, lifetime information, service history, or sterilization history. In any of the preceding embodiments, the controller is further configured to determine a parameter regarding the end effector. In some embodiments, the parameter is retrieved from a database. In some embodiments, the parameter is detected by the controller. In some embodiments, the parameter is at least one of a version, a class, manufacturing information, shape, a dimension, weight, center of mass, duration of use, lifetime information, service history, or sterilization history. In any of the preceding embodiments, the controller is further configured to determine a parameter regarding the end effector before the end effector is coupled to the robot arm. In any of the preceding embodiments, wherein the controller is further configured to determine a parameter regarding the end effector after the end effector is coupled to the robot arm. In any of the preceding embodiments, the operation adjustment is at least one of an adjustment to the robot arm based on an estimated load and/or force exerted by the end effector weight or center of mass, an adjustment to the robot arm to allow for optimal joint configuration of the robot arm based on tool trajectory, or an adjustment to calibration data. In some embodiments, the adjustment to calibration data reflects a dimension of the end effector. In any of the preceding embodiments, the controller is further configured to determine manufacturing information, obtain one or more of end effector or tool geometry, and adjust the robot arm to improve accuracy. In some embodiments, the tool geometry is at least one of a distance of a tool from a distal end of the robot arm, an angle of a tool axis relative to the distal end of the robot arm, or a depth a tool tip will protrude from the end effector. In any of the preceding embodiments, the controller is further configured to determine at least one of use, service, or replacement data for the end effector. In some embodiments, the controller is further configured to determine if a lifetime maximum duration of use will be exceeded during a procedure. In any of the preceding embodiments, the controller is further configured to update data stored in the memory of the end effector. In some embodiments, the updated data is based on at least one of use of the end effector, a unique identifier of the robot arm to which the end effector was coupled, a most recently performed procedure, sterilization history, or service update.

In a second example, an end effector comprises a memory for storing data about the end effector; and a processor configured to send the data to a controller for a robotic surgical system. In some embodiments, the data sent from the end effector is at least one of a unique identifier, a version identifier, a class identifier, manufacturing information, shape, a dimension, weight, center of mass, duration of use, lifetime information, service history, or sterilization history. In any of the preceding embodiments, the processor is further configured to receive updated data from the controller to replace the data stored in memory. In some embodiments, the updated data is based on at least one of use of the end effector, a unique identifier of a robot arm to which the end effector was coupled, a most recently performed procedure, sterilization history, or service update.

In a third example, a surgical method comprises coupling an end effector to a robot arm that is coupled to a controller; identifying the end effector with the controller using data received from the end effector; and adjusting operation of the surgical system (for example, the robot arm) with the controller based on the data received from the end effector. In some embodiments, the data received from the end effector includes end effector weight. In any of the preceding embodiments, the end effector weight is a parameter stored in a memory of the end effector and communicated to the controller. In any of the preceding embodiments, the end effector weight is detected with the controller from one or more mechanical loads placed on the robot arm when the end effector is coupled to the robot arm. In any of the preceding embodiments, the data received from the end effector includes end effector center of mass. In any of the preceding embodiments, the data received from the end effector includes a dimensional parameter of the end effector. In some embodiments, the dimensional parameter includes a distance of a tool from a distal end of the robot arm. In some embodiments, the dimensional parameter includes an angle of a tool axis relative to a distal end of the robot arm. In any of the preceding embodiments, the data received from the end effector includes a unique identifier stored in a memory of the end effector and communicated to the controller. In any of the preceding embodiments, the data received from the end effector includes its shape as recognized by one or more cameras coupled to the controller. In any of the preceding embodiments, the data received from the end effector includes a number of procedures in which the end effector was used. In any of the preceding embodiments, the data received from the end effector includes a number of times the end effector has been attached to a robot arm. In any of the preceding embodiments, the data received from the end effector includes a listing of robot arm identifiers for each robot arm to which the end effector has been attached. In any of the preceding embodiments, the data received from the end effector includes a duration of use. In some embodiments, the duration of use is a lifetime sum. In some embodiments, the method further comprises determining if the duration of use of the end effector is greater than a lifetime maximum. In some embodiments, the method further comprises determining if a lifetime maximum duration of use will be exceeded during a procedure. In some embodiments, the duration of use is listed for each attachment to a robot arm. In any of the preceding embodiments, the data received from the end effector includes manufacturing information. In some embodiments, the method further comprises providing feedback to a user based on the manufacturing information. In any of the preceding embodiments, the data received from the end effector includes tool trajectory information to allow for optimal joint configuration of the robot arm. In any of the preceding embodiments, the adjusting operation of the robot arm includes implementing a control software module based on the data received from the end effector. In any of the preceding embodiments, the adjusting operation of the robot arm includes implementing a control algorithm to optimize operation of the robot arm based on estimated loads from the end effector. In any of the preceding embodiments, the method further comprises displaying to a user a parameter based on the data received from the end effector. In any of the preceding embodiments, the parameter displayed to the user includes at least one of lifetime information, service history, or sterilization history. In any of the preceding embodiments, the method further comprises communicating with a remote processor over a network to track any of use, service, or replacement of the end effector. In any of the preceding embodiments, the method further comprises updating data stored in a memory of the end effector based on use of the end effector.

1. A robotic surgical system, comprising:
a robot arm;
a controller operably connected to the robot arm for positioning a tool with respect to a patient, wherein the tool is retained by an end effector, the controller being configured to:
determine whether the end effector is coupled to the robot arm;
receive data from the end effector;
identify the end effector; and
adjust operation of the surgical system based on the data received from the end effector.

2. The system of claim 1, wherein the data received from the end effector is at least one of a unique identifier, a version identifier, a class identifier, manufacturing information, shape, a dimension, weight, center of mass, duration of use, lifetime information, service history, or sterilization history.

3. The system of claim 1, wherein the controller is further configured to determine a parameter regarding the end effector.

4. The system of claim 3, wherein the parameter is retrieved from a database.

5. The system of claim 3, wherein the parameter is detected by the controller.

6. The system of claim 3, wherein the parameter is at least one of a version, a class, manufacturing information, shape, a dimension, weight, center of mass, duration of use, lifetime information, service history, or sterilization history.

7. The system of claim 1, wherein the controller is further configured to determine a parameter regarding the end effector before the end effector is coupled to the robot arm.

8. The system of claim 1, wherein the controller is further configured to determine a parameter regarding the end effector after the end effector is coupled to the robot arm.

9. The system of claim 1, wherein the operation adjustment is at least one of an adjustment to the robot arm based on an estimated load and/or force exerted by the end effector weight or center of mass, an adjustment to the robot arm to allow for optimal joint configuration of the robot arm based on tool trajectory, or an adjustment to calibration data.

10. The system of claim 9, wherein the adjustment to calibration data reflects a dimension of the end effector.

11. The system of claim 1, wherein the controller is further configured to determine manufacturing information, obtain one or more of end effector or tool geometry, and adjust the robot arm to improve accuracy.

12. The system of claim 11, wherein the tool geometry is at least one of a distance of a tool from a distal end of the robot arm, an angle of a tool axis relative to the distal end of the robot arm, or a depth a tool tip will protrude from the end effector.

13. The system of claim 1, wherein the controller is further configured to determine at least one of use, service, or replacement data for the end effector.

14. The system of claim 13, wherein the controller is further configured to determine if a lifetime maximum duration of use will be exceeded during a procedure.

15. The method of claim 1, wherein the controller is further configured to update data stored in the memory of the end effector.

16. The system of claim 15, wherein the updated data is based on at least one of use of the end effector, a unique identifier of the robot arm to which the end effector was coupled, a most recently performed procedure, sterilization history, or service update.

17. An end effector, comprising:
a memory for storing data about the end effector; and
a processor configured to send the data to a controller for a robotic surgical system.

18. The end effector of claim 18, wherein the data sent from the end effector is at least one of a unique identifier, a version identifier, a class identifier, manufacturing information, shape, a dimension, weight, center of mass, duration of use, lifetime information, service history, or sterilization history.

19. The end effector of claim 18, wherein the processor is further configured to receive updated data from the controller to replace the data stored in memory.

20. The end effector of claim 19, wherein the updated data is based on at least one of use of the end effector, a unique identifier of a robot arm to which the end effector was coupled, a most recently performed procedure, sterilization history, or service update.
